SEQUENCE LISTING (8 sequences)

SEQ ID NO: 1 - protein, 118 aa, Homo sapiens
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 2 - protein, 108 aa, Homo sapiens
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 3 - protein, 118 aa, Homo sapiens
QVQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 4 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

SEQ ID NO: 5 - DNA, 990 bp, Homo sapiens
  CDS (1)..(990)
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 330 aa, Homo sapiens [sequence omitted]

SEQ ID NO: 7 - DNA, 321 bp, Homo sapiens
  CDS (1)..(321)
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcacaggagagcgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 107 aa, Homo sapiens [sequence omitted]

(19) United States
(12) Patent Application Publication
(10) Pub. No.: US 2008/0226635 A1
(43) Pub. Date: Sep. 18, 2008

(54) Antibodies against insulin-like growth factor I receptor and uses thereof

(76) Inventors: Hans Koll, Oberroth (DE); Klaus-Peter Kuenkele, Benediktbeuern (DE); Samuel Moser, Zuerich (CH); Sylke Poehling, Wolfratshausen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/001,332

(22) Filed: Dec. 11, 2007

(30) Foreign Application Priority Data
Dec. 22, 2006 (EP) 06026651.7

(51) Int. Cl. A61K 39/395; C07K 16/18; C12N 5/06; A61P 43/00

(52) U.S. Cl. 424/138.1; 530/387.1; 530/387.3; 435/330

(57) ABSTRACT

An antibody binding to IGF-IR and being glycosylated with a sugar chain at Asn297, said antibody being characterized in that the amount of fucose within said sugar chain is between 20% and 50%, has improved properties in antitumor therapy.

### PRIORITY TO RELATED APPLICATION(S)

This application claims the benefit of European Patent Application No. 06026651.7, filed Dec. 22, 2006, which is hereby incorporated by reference in its entirety.

The present invention relates to antibodies against human insulin-like growth factor I receptor (IGF-IR), methods for their production, pharmaceutical compositions containing said antibodies, and uses thereof.

Insulin-like growth factor I receptor (IGF-IR, EC 2.7.112, CD 221 antigen) belongs to the family of transmembrane protein tyrosine kinases (LeRoith, D., et al., Endocrin. Rev. 16 (1995) 143-163; and Adams, T. E., et al., Cell. Mol. Life. Sci. 57 (2000) 1050-1093). IGF-IR binds IGF-I with high affinity and initiates the physiological response to this ligand in vivo. IGF-IR also binds to IGF-II, however with slightly lower affinity. The IGF-1 system, including IGF-1R, plays an important role during proliferation of (normal and neoplastic) cells. IGF-1R is found on normal human tissues e.g. from placenta, prostate, bladder, kidney, duodenum, small bowel, gallbladder, common bile duct, intrahepatic bile duct, bronchi, tonsil, thymus, breast, sebaceous gl., salivary gland, uterine cervix, and salpinx. IGF-IR overexpression promotes the neoplastic transformation of cells and there exists evidence that IGF-IR is involved in malignant transformation of cells and is therefore a useful target for the development of therapeutic agents for the treatment of cancer (Adams, T. E., et al., Cell. Mol. Life. Sci. 57 (2000) 1050-1093).

Antibodies against IGF-IR are well-known in the state of the art and investigated for their antitumor effects in vitro and in vivo (Benini, S., et al., Clin. Cancer Res. 7 (2001) 1790-1797; Scotlandi, K., et al., Cancer Gene Ther. 9 (2002) 296-307; Scotlandi, K., et al., Int. J. Cancer 101 (2002) 11-16; Brunetti, A., et al., Biochem. Biophys. Res. Commun. 165 (1989) 212-218; Prigent, S. A., et al., J. Biol. Chem. 265 (1990) 9970-9977; Li, S. L., et al., Cancer Immunol. Immunother. 49 (2000) 243-252; Pessino, A., et al., Biochem. Biophys. Res. Commun. 162 (1989) 1236-1243; Surinya, K. H., et al., J. Biol. Chem. 277 (2002) 16718-16725; Soos, M. A., et al., J. Biol. Chem., 267 (1992) 12955-12963; Soos, M. A., et al., Proc. Natl. Acad. Sci. USA 86 (1989) 5217-5221; O'Brien, R. M., et al., EMBO J. 6 (1987) 4003-4010; Taylor, R., et al., Biochem. J. 242 (1987) 123-129; Soos, M. A., et al., Biochem. J. 235 (1986) 199-208; Li, S. L., et al., Biochem. Biophys. Res. Commun. 196 (1993) 92-98; Delafontaine, P., et al., J. Mol. Cell. Cardiol. 26 (1994) 1659-1673; Kull, F. C. Jr., et al. J. Biol. Chem. 258 (1983) 6561-6566; Morgan, D. O., and Roth, R. A., Biochemistry 25 (1986) 1364-1371; Forsayeth, J. R., et al., Proc. Natl. Acad. Sci. USA 84 (1987) 3448-3451; Schaefer, E. M., et al., J. Biol. Chem. 265 (1990) 13248-13253; Gustafson, T. A., and Rutter, W. J., J. Biol. Chem. 265 (1990) 18663-18667; Hoyne, P. A., et al., FEBS Lett. 469 (2000) 57-60; Tulloch, P. A., et al., J. Struct. Biol. 125 (1999) 11-18; Rohlik, Q. T., et al., Biochem. Biophys. Res. Comm. 149 (1987) 276-281; and Kalebic, T., et al., Cancer Res. 54 (1994) 5531-5534; Adams, T. E., et al., Cell. Mol. Life. Sci. 57 (2000) 1050-1093; Dricu, A., et al., Glycobiology 9 (1999) 571-579; Kanter-Lewensohn, L., et al., Melanoma Res. 8 (1998) 389-397; Li, S. L., et al., Cancer Immunol. Immunother. 49 (2000) 243-252). Antibodies against IGF-IR are also described in a lot of further publications, e.g., Arteaga, C. L., et al., Breast Cancer Res. Treatment 22 (1992) 101-106; and Hailey, J., et al., Mol. Cancer. Ther. 1 (2002) 1349-1353.

In particular, the monoclonal antibody against IGF-IR called αIR3 is widely used in the investigation of studying IGF-IR mediated processes and IGF-I mediated diseases such as cancer. Alpha-IR-3 was described by Kull, F. C., J. Biol. Chem. 258 (1983) 6561-6566. In the meantime, about a hundred publications have been published dealing with the investigation and therapeutic use of αIR3 in regard to its antitumor effect, alone and together with cytostatic agents such as doxorubicin and vincristine. αIR3 is a murine monoclonal antibody which is known to inhibit IGF-I binding to IGF receptor but not IGF-II binding to IGF-IR. αIR3 stimulates at high concentrations tumor cell proliferation and IGF-IR phosphorylation (Bergmann, U., et al., Cancer Res. 55 (1995) 2007-2011; Kato, H., et al., J. Biol. Chem. 268 (1993) 2655-2661). There exist other antibodies (e.g., 1H7, Li, S. L., et al., Cancer Immunol. Immunother. 49 (2000) 243-252) which inhibit IGF-II binding to IGF-IR more potently than IGF-I binding. A summary of the state of the art of antibodies and their properties and characteristics is described by Adams, T. E., et al., Cell. Mol. Life. Sci. 57 (2000) 1050-1093.

Most of the antibodies described in the state of the art are of mouse origin. Such antibodies are, as is well known in the state of the art, not useful for the therapy of human patients without further alterations like chimerization or humanization. Based on these drawbacks, human antibodies are clearly preferred as therapeutic agents in the treatment of human patients. Human antibodies are well-known in the state of the art (van Dijk, M. A., and van de Winkel, J. G., Curr. Opin. Chem. Biol. 5 (2001) 368-374). Based on such technology, human antibodies against a great variety of targets can be produced. Examples of human antibodies against IGF-IR are described in WO 2002/053596, WO 2004/071529, WO 2005/016967, WO 2006/008639, US 2005/0249730, US 2005/0084906, WO 2005/058967, WO 2006/013472, US 2003/0165502, WO 2005/082415, WO 2005/016970, WO 2003/106621, WO 2004/083248, WO 2003/100008, WO 2004/087756, WO 2005/005635 and WO 2005/094376.

WO 2004/087756 describes antibodies binding to IGF-1R and inhibiting the binding of IGF-I and IGF-II to IGF-1R characterized in being of human IgG1 isotype, and showing a ratio of inhibition of the binding of IGF-I to IGF-IR to the inhibition of binding of IGF-II to IGF-IR of 1:3 to 3:1, and induces cell death of 20% or more cells of a preparation of IGF-IR expressing cells after 24 hours at a concentration of said antibody of 100 nM by ADCC.

Cell-mediated effector functions of monoclonal antibodies can be enhanced by engineering their oligosaccharide component as described in Umaña, P., et al., Nature Biotechnol. 17 (1999) 176-180; and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A. and Morrison, S. L., Trends Biotechnol. 15 (1997) 26-32). Umaña, P., et al. Nature Biotechnol. 17 (1999) 176-180 and WO 99/54342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the N297 carbohydrate or its elimination affect also binding to Fc binding to FcγR and Cl q (Umaña, P., et al., Nature Biotechnol. 17 (1999) 176-180; Davies et al., Biotechnol. Bioeng. 74 (2001) 288-294; Mimura, Y., et al., J. Biol. Chem. 276 (2001) 45539-45547; Radaev et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Iida, S., et al., Clin. Cancer Res. 12 (2006) 2879-2887 show that efficacy of a nonfucosylated anti-CD20 antibody was inhibited by addition of fucosylated anti-CD20. The efficacy of a 1:9 mixture (10 microg/mL) of nonfucosylated and fucosylated anti-CD20s was inferior to that of a 1,000-fold dilution (0.01 microg/mL) of nonfucosylated anti-CD20 alone. They conclude that nonfucosylated IgG1, not including fucosylated counterparts, can evade the inhibitory effect of plasma IgG on ADCC through its high FcgammaRIIIa binding. Natsume, A., et al., shows in J. Immunol. Methods 306 (2005) 93-103 that Fucose removal from complex-type oligosaccharide of human IgG1-type antibody results in a great enhancement of antibody-dependent cellular cytotoxicity (ADCC). Satoh, M., et al., Expert Opin. Biol. Ther. 6 (2006) 1161-1173 discusses non-fucosylated therapeutic antibodies as next-generation therapeutic antibodies. Satoh concludes that antibodies consisting of only the non-fucosylated human IgG1 form is thought to be ideal. Kanda, Y., et al., Biotechnol. Bioeng. 94 (2004) 680-688 compared fucosylated CD20 antibody (96% fucosylation, CHO/DG44 1H5) with non-fucosylated CD20 antibody. Davies, J., et al., Biotechnol. Bioeng. 74 (2001) 288-294 reports that for a CD20 antibody increased ADCC correlates with increased binding to FcγRIII.

Methods to enhance cell-mediated effector functions of monoclonal antibodies are described e.g. in WO 2005/018572, WO 2006/116260, WO 2006/114700, WO 2004/065540, WO 2005/011735, WO 2005/027966, WO 1997/028267, US 2006/0134709, US 2005/0054048, US 2005/0152894, WO 2003/035835, WO 2000/061739.

There is still a need for antibodies against IGF-IR with convincing benefits for patients in need of antitumor therapy. The relevant benefit for the patient is, in simple terms, reduction in tumor growth and a significant prolongation of time to progression caused by the treatment with the antitumorigenic agent.

### SUMMARY OF THE INVENTION

The invention comprises an antibody binding to IGF-IR, and being glycosylated with a sugar chain at Asn297, said antibody being characterized in that the amount of fucose within said sugar chain is between 20% and 50%, preferably between 20% and 40%.

Antibodies according to the invention comprising such amount of fucose are further termed as partially fucosylated.

The invention comprises an antibody binding to IGF-IR and being glycosylated with a sugar chain at Asn297, said antibody being characterized in showing high binding affinity to the FcγRIII.

Preferably the antibody is of human IgG1, IgG2, IgG3 or IgG4 type. More preferably the antibody is of human IgG1 or IgG3 type. Most preferably, the antibody is a human IgG1 type.

Preferably the amount of N-glycolylneuraminic acid (“NGNA”) is 1% or less and/or the amount of N-terminal alpha-1,3-galactose is 1% or less.

Preferably the amount of NGNA is 0.5% or less, more preferably 0.1% or less and even not detectable (LCMS).

Preferably the amount of N-terminal alpha-1,3-galactose is 0.5% or less, more preferably 0.1% or less and even not detectable (LCMS).

According to the invention “amount of fucose” means the amount of said sugar within the sugar chain at Asn297, related to the sum of all glycostructures attached to Asn 297 (e.g. complex, hybrid and high mannose structures) measured by MALDI-TOF mass spectrometry and calculated as average value (see example 4).

The sugar chain show preferably the characteristics of N-linked glycans attached to Asn297 of an antibody binding to IGF-IR recombinantly expressed in a CHO cell.

The invention comprises preferably a partially fucosylated antibody binding to IGF-IR and inhibiting the binding of IGF-I and IGF-II to IGF-IR, characterized in that said antibody preferably shows one or more properties selected from the group consisting of:
a) shows a ratio of IC₅₀ values of inhibition of the binding of IGF-I to IGF-IR to the inhibition of binding of IGF-II to IGF-IR of 1:3 to 3:1,
b) inhibits for at least 80%, preferably at least 90%, at a concentration of 5 nM IGF-IR phosphorylation in a cellular phosphorylation assay using HT29 cells in a medium containing 0.5% heat inactivated fetal calf serum (FCS) and 10 nM human IGF-1, when compared to such an assay without said antibody.
c) shows no IGF-IR stimulating activity (no signaling, no IGF-1 mimetic activity) measured as PKB phosphorylation at a concentration of 10 μM in a cellular phosphorylation assay using 3T3 cells providing 400,000 to 600,000 molecules IGF-IR per cell in a medium containing 0.5% heat inactivated fetal calf serum (FCS) when compared to such an assay without said antibody.

Antibodies according to the invention show benefits for patients in need of antitumor therapy and provide reduction of tumor growth and a significant prolongation of the time to progression. The antibodies according to the invention have new and inventive properties causing a benefit for a patient suffering from a disease associated with an IGF deregulation, especially a tumor disease. The antibodies according to the invention are characterized by the above mentioned properties.

Preferably the antibody binds specifically to IGF-IR, inhibits the binding of IGF-I and IGF-II to IGF-IR at the abovementioned ratio, is of IgG1 isotype and is partially fucosylated, and does not activate IGF-IR signaling even in IGF-IR overexpressing cells at a 200-fold concentration or even 20.000-fold concentration of its IC₅₀ value.

Preferably, at a concentration of 5 nM the antibodies according to the invention completely inhibit IGF-I mediated signal transduction of IGF-IR in tumor cells.

The antibody is preferably a monoclonal antibody and, in addition, a chimeric antibody (human constant chain), a humanized antibody and especially preferably a human antibody.

The antibody preferably binds to IGF-IR human (EC 2.7.1.112, SwissProt P08069) in competition to antibody 18.

The antibody is preferably further characterized by an affinity of 10⁻⁸ M (K_{D}) or less, preferably of about 10⁻⁹ to 10⁻¹³ M.

The antibody shows preferably no detectable concentration dependent inhibition of insulin binding to the insulin receptor.

The antibody is preferably of IgG1 type.

The antibody according to the invention considerably prolongates the time to progression in relevant xenograft tumor models in comparison with vehicle treated animals and reduces tumor growth. The antibody inhibits the binding of IGF-I and IGF-II to IGF-IR in vitro and in vivo, preferably in about an equal manner for IGF-I and IGF-II.

Preferably, the antibodies according to the invention comprise as heavy chain complementarity determining region CDR3 a sequence selected from the group consisting of SEQ ID NO:1 or 3. Preferably, the antibodies according to the invention comprise as complementarity determining regions (CDRs) the following sequences:
a) an antibody heavy chain comprising as CDRs CDR1 (aa 31-35), CDR2 (aa 50-66) and CDR3 (aa 99-107) of SEQ ID NO:1 or 3;
b) an antibody light chain comprising as CDRs CDR1 (aa 24-34), CDR2 (aa 50-56) and CDR3 (aa 89-98) of SEQ ID NO:2 or 4.

Preferred variable regions and CDRs, especially CDR3 of heavy chain of antibodies according to the invention are provided by <IGF-1R> HUMAB Clone 18 (antibody 18) and <IGF-1R> HUMAB Clone 22 (antibody 22), deposited under the Budapest Treaty with Deutsche Sammlung von Mikroorganismen und Zellkulturen GmbH (DSMZ), Inhoffenstrasse 7 B, 38124 Braunschweig, Germany.

| Cell line | Deposition No. | Date of deposit |
|---|---|---|
| <IGF-1R> HUMAB-Clone 18 | DSM ACC 2587 | Oct. 04, 2003 |
| <IGF-1R> HUMAB-Clone 22 | DSM ACC 2594 | Sep. 05, 2003 |

Preferably, the antibody according to the invention comprises an antibody heavy chain comprising SEQ ID NO:1 and an antibody light chain comprising SEQ ID NO:2 or an antibody heavy chain comprising SEQ ID NO:3 and an antibody light chain SEQ ID NO:4. The antibody is preferably of human IgG1 type.

These antibodies are described in detail in WO 2005/005635.

The invention further provides methods for the recombinant production of such antibodies. Preferred nucleic acids of polypeptides which are capable of assembling together with the respective other antibody chain to an antibody according to the invention are defined below:
a) an antibody heavy chain comprising as CDRs CDR1 (aa 31-35), CDR2 (aa 50-66) and CDR3 (aa 99-107) of SEQ ID NO:1 or 3;
b) an antibody light chain comprising as CDRs CDR1 (aa 24-34), CDR2 (aa 50-56) and CDR3 (aa 89-98) of SEQ ID NO:2 or 4.

Preferably, the nucleic acids encode an antibody according to the invention which comprise an antibody heavy chain comprising of SEQ ID NO:1 and an antibody light chain of SEQ ID NO:2 or an antibody heavy chain comprising of SEQ ID NO:3 and an antibody light chain of SEQ ID NO:4.

The invention further provides methods for treating cancer, preferably breast cancer, pancreatic cancer, prostate cancer, bladder cancer, malignal melanoma, Ewing's sarcoma, Neuroblastoma, Osteosarcoma, Rhabdomyosarcoma, and/or NSCLC, comprising administering to a patient diagnosed as having cancer (and therefore being in need of an antitumor therapy) an antibody against IGF-IR according to the invention. The antibody may be administered alone, in a pharmaceutical composition, or alternatively in combination with other inhibitors of cancer related signaling pathways such as EGFR, Her2/neu or estrogen receptor or in combination with a cytotoxic treatment such as radiotherapy or a cytotoxic agent or a prodrug thereof. The antibody is administered in a pharmaceutically effective amount.

The invention further comprises the use of an antibody according to the invention for cancer treatment, preferably breast cancer, pancreatic cancer and/or NSCLC, and for the manufacture of a pharmaceutical composition according to the invention. In addition, the invention comprises a method for the manufacture of a pharmaceutical composition according to the invention.

The invention further comprises an antibody according to the invention for cancer treatment, preferably breast cancer, pancreatic cancer, prostate cancer, bladder cancer, malignal melanoma, Ewing's sarcoma, Neuroblastoma, Osteosarcoma, Rhabdomyosarcoma and/or NSCLC.

The invention further comprises a pharmaceutical composition containing an antibody according to the invention, optionally together with a buffer and/or an adjuvant useful for the formulation of antibodies for pharmaceutical purposes.

The invention further comprises a pharmaceutical composition comprising an antibody according to the invention.

The invention further provides pharmaceutical compositions comprising such antibodies in a pharmaceutically acceptable carrier. In one embodiment, the pharmaceutical composition may be included in an article of manufacture or kit. The invention further provides the use of an antibody according to the invention for the manufacture of a pharmaceutical composition for the treatment of cancer. The antibody is used in a pharmaceutically effective amount.

The invention further comprises the use of an antibody according to the invention for the manufacture of a pharmaceutical composition for the treatment of cancer, preferably breast cancer, pancreatic cancer and/or NSCLC. The antibody is used in a pharmaceutically effective amount.

The invention further comprises a method for the production of a recombinant human antibody according to the invention, characterized by expressing a nucleic acid encoding an antibody binding to IGF-1R in a CHO host cell, which partially fucosylates said antibody and recovering said antibody from said cell. The invention further comprises the antibody obtainable by such a recombinant method.

The invention further comprises a CHO cell capable of recombinantly expressing GnTIII and an anti-IGF-1R antibody. Such a CHO cell is a CHO cell, transformed with a first DNA sequence encoding a polypeptide having GnTIII activity, a second DNA sequence encoding at least the variable domain of the heavy chain, and a third DNA sequence encoding at least the variable domain of the light chain of an antibody against IGF-IR. Preferably the second and third DNA sequences encode the heavy and light chain of an antibody against IGF-IR of human IgG1 type.

The invention further comprises a process for the production of an antibody against IGF-1R, comprising the steps of transforming a host cell, preferably a CHO cell, with a first DNA sequence encoding a polypeptide having GnTIII activity, a second DNA sequence encoding at least the variable domain of the heavy chain, and a third DNA sequence encoding at least the variable domain of the light chain of an antibody against IGF-IR, cultivating in a fermentation medium said host cell, which expresses, preferably independently, said first, second and third DNA sequences, under conditions that said host cell secretes said antibody to the fermentation medium and isolating said antibody.

### BRIEF DESCRIPTION OF THE DRAWINGS

FIG. 1 shows the MALDI-MS-analysis of released oligosaccharides of samples 1 and 2.

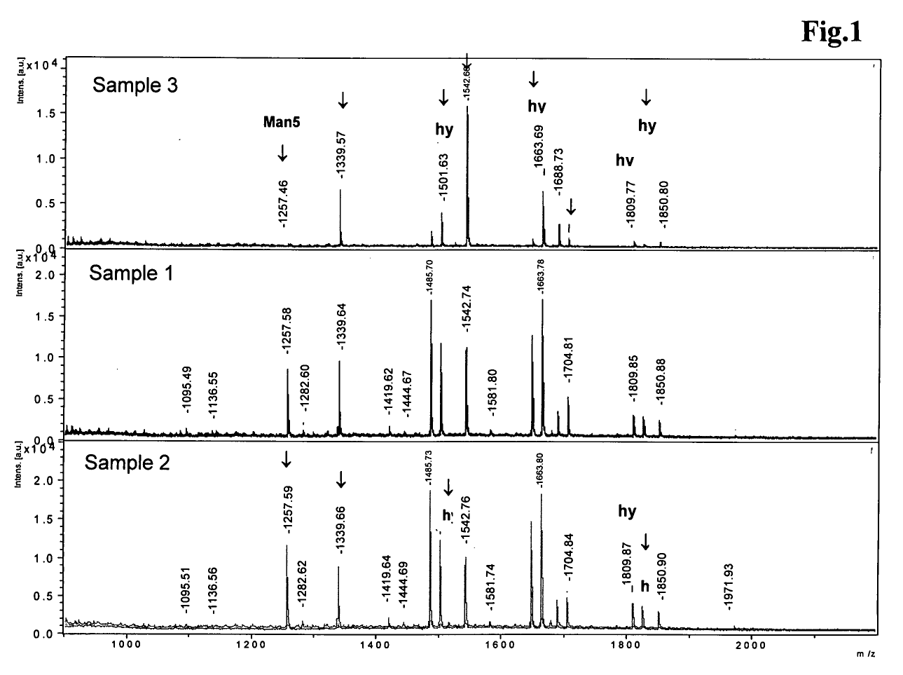

FIG. 2 shows tumor volume measured according to example 3 (curve 1: control).

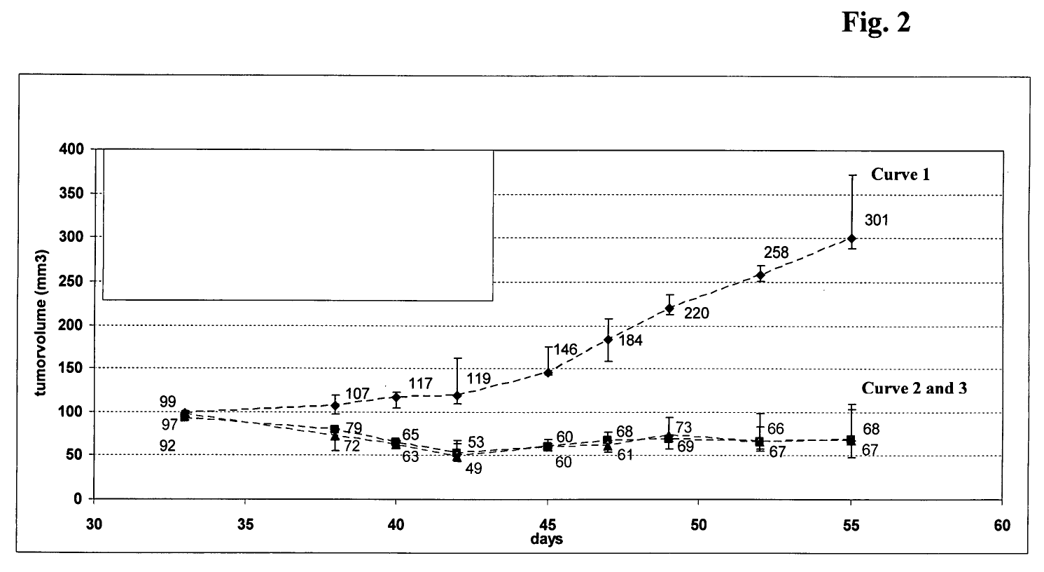

### DETAILED DESCRIPTION OF THE INVENTION

The term “antibody” encompasses the various forms of antibodies including but not being limited to whole antibodies, antibody fragments, human antibodies, humanized antibodies and genetically engineered antibodies as long as the characteristic properties according to the invention are retained.

“Antibody fragments” comprise a portion of a full length antibody, generally at least the antigen binding portion or the variable region thereof. Examples of antibody fragments include diabodies, single-chain antibody molecules, immunotoxins, and multispecific antibodies formed from antibody fragments.

The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric and bifunctional antibodies.

The term “human antibody”, as used herein, is intended to include antibodies having variable and constant regions derived from human germline immunoglobulin sequences. The variable heavy chain is preferably derived from germline sequence DP-50 (GenBank LO6618) and the variable light chain is preferably derived from germline sequence L6 (GenBank X01668) or the variable heavy chain is preferably derived DP-61 (GenBank M99682) and the variable light chain is derived from germline sequence L15 (GenBank K01323). The constant regions of the antibody are constant regions of human IgG1 type. Such regions can be allotypic and are described by, e.g., Johnson, G., and Wu, T. T., Nucleic Acids Res. 28 (2000) 214-218 and the databases referenced therein.

The term “recombinant human antibody”, refers to antibodies having variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

As used herein, “binding” refers to antibody binding to IGF-IR with an affinity of about 10⁻¹³ to 10⁻⁸ M (K_{D}), preferably of about 10⁻¹³ to 10⁻⁹ M.

The term “nucleic acid molecule”, as used herein, is intended to include DNA molecules and RNA molecules. A nucleic acid molecule may be single-stranded or double-stranded, but preferably is double-stranded DNA.

Human constant domains having of IgG1 or IgG3 type are described in detail by Kabat, E. A., et al., Sequences of Proteins of Immunological Interest, 5th ed., Public Health Service, National Institutes of Health, Bethesda, Md. (1991), and by Brueggemann, M., et al., J. Exp. Med. 166 (1987) 1351-1361; Love, T. W., et al., Methods Enzymol. 178 (1989) 515-527. Examples are shown in SEQ ID NOS:5 to 8. Other useful and preferred constant domains are the constant domains of the antibodies obtainable from the hybridoma cell lines deposited with DSMZ for this invention.

Constant domains of human IgG1, IgG2 or IgG3 type are glycosylated at Asn297. “Asn 297” according to the invention means amino acid asparagine located at about position 297 in the Fc region; based on minor sequence variations of antibodies, Asn297 can also be located some amino acids (usually not more than ±3 amino acids) upstream or downstream. For example, in one antibody according to the invention (AK18) “Asn297” is located at amino acid position 298.

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding and characterizes the antibody. CDR and FR regions are determined according to the standard definition of Kabat, E. A., et al., supra.

Glycosylation of human IgG1 or IgG3 occurs at Asn297 as core fucosylated bianntennary complex oligosaccharide glycosylation terminated with up to 2 Gal residues. These structures are designated as G0, G1 (α1,6 or α1,3) or G2 glycan residues, depending from the amount of terminal Gal residues (Raju, T. S., BioProcess Int. 1 (2003) 44-53). CHO type glycosylation of antibody Fc parts is e.g. described by Routier, F. H., Glycoconjugate J. 14 (1997) 201-207. Antibodies which are recombinantly expressed in non glycomodified CHO host cells usually are fucosylated at Asn297 in an amount of at least 85%.

The partially fucosylated IGF-1R antibody according to the invention can be expressed in a glycomodified host cell engineered to express at least one nucleic acid encoding a polypeptide having GnTIII activity in an amount sufficient to partially fucosylate the oligosaccharides in the Fc region. In one embodiment, the polypeptide having GnTIII activity is a fusion polypeptide. Alternatively α1,6-fucosyltransferase activity of the host cell can be decreased or eliminated according to U.S. Pat. No. 6,946,292 to generate glycomodified host cells. The amount of antibody fucosylation can be predetermined e.g. either by fermentation conditions (e.g. fermentation time) or by combination of at least two antibodies with different fucosylation amount.

The IGF-1R antibody according to the invention can be produced in a host cell by a method comprising: (a) culturing a host cell engineered to express at least one polynucleotide encoding a fusion polypeptide having GnTIII activity under conditions which permit the production of said antibody with partial fucosylation of the oligosaccharides present on the Fc region of said antibody; and (b) isolating said antibody. In one embodiment, the polypeptide having GnTIII activity is a fusion polypeptide, preferably comprising the catalytic domain of GnTIII and the Golgi localization domain of a heterologous Golgi resident polypeptide selected from the group consisting of the localization domain of mannosidase II, the localization domain of β(1,2)-N-acetylglucosaminyltransferase I (“GnTI”), the localization domain of mannosidase I, the localization domain of β(1,2)-N-acetylglucosaminyltransferase II (“GnTII”), and the localization domain of α1-6 core fucosyltransferase. Preferably, the Golgi localization domain is from mannosidase II or GnTI. In a further aspect, the invention is directed to a method for modifying the glycosylation profile of an anti-IGF-1R antibody by using such method.

In another aspect, the present invention is directed to a method of modifying the glycosylation of an IGF-1R antibody by using a fusion polypeptide having GnTIII activity and comprising the Golgi localization domain of a heterologous Golgi resident polypeptide. In one embodiment, the fusion polypeptides of the invention comprise the catalytic domain of GnTIII In another embodiment, the Golgi localization domain is selected from the group consisting of: the localization domain of mannosidase II, the localization domain of GnTI, the localization domain of mannosidase I, the localization domain of GnTII and the localization domain of α1-6 core fucosyltransferase. Preferably, the Golgi localization domain is from mannosidase II or GnTI.

According to the present invention, these modified oligosaccharides of the IGF-1R antibody may be hybrid or complex. Preferably the bisected, nonfucosylated oligosaccharides are hybrid. In another embodiment, the bisected, nonfucosylated oligosaccharides are complex.

As used herein, a polypeptide having GnTIII activity refers to polypeptides that are able to catalyze the addition of a N-acetylglucosamine (GlcNAc) residue in β-1-4 linkage to the β-linked mannoside of the trimannosyl core of N-linked oligosaccharides. This includes fusion polypeptides exhibiting enzymatic activity similar to, but not necessarily identical to, an activity of β(1,4)-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependence in a given activity as compared to the GnTIII (i.e., the candidate polypeptide will exhibit greater activity or not more than about 25-fold less and, preferably, not more than about tenfold less activity, and most preferably, not more than about three-fold less activity relative to the GnTIII.) As used herein, the term Golgi localization domain refers to the amino acid sequence of a Golgi resident polypeptide which is responsible for anchoring the polypeptide in location within the Golgi complex. Generally, localization domains comprise amino terminal “tails” of an enzyme.

The antibodies according to the invention show high binding affinity to the FcyRIII (CD16a). High binding affinity to FcγRIII denotes that binding is enhanced for CD16a/F158 at least 10-fold in relation to the wt antibody (95% fucosylation) as standard (see example 5) expressed in CHO DG44 host cells and binding is enhanced for CD16a/V158 at least 20-fold in relation to the wt antibody measured by Surface Plasmon Resonance (SPR) using immobilized CD16a at an antibody concentration of 100 nM (see example 5). FcγRIII binding can be increased by methods according to the state of the art by modifying the amino acid sequence of the Fc part or the glycosylation of the Fc part of the antibody. Preferred methods are described above.

The term “binding to IGF-IR” as used herein means the binding of the antibody to IGF-IR in an in vitro assay, preferably in a binding assay in which the antibody is bound to a surface and binding of IGF-IR is measured by Surface Plasmon Resonance (SPR). Binding means a binding affinity (K_{D}) of 10⁻⁸ M or less, preferably 10⁻¹³ to 10⁻⁹ M.

Binding of the antibody to IGF-1R or FcγRIII can be investigated by a BIAcore assay (Pharmacia Biosensor AB, Uppsala, Sweden). The affinity of the binding is defined by the terms ka (rate constant for the association to the target antibody), kd (dissociation constant), and K_{D} (kd/ka). The antibodies according to the invention show a K_{D} of 10⁻⁹ M or less, preferably a K_{D} of 10⁻¹⁰ M or less for the binding to IGF-1R.

The binding of IGF-I and IGF-II to IGF-IR is also inhibited by the antibodies according to the invention. The inhibition is measured as IC₅₀ in an assay for binding of IGF-I/IGF-II to IGF-IR on tumor cells. In such an assay, the amount of radiolabeled IGF-I or IGF-II or IGF-IR binding fragments thereof bound to the IGF-IR provided at the surface of said tumor cells (e.g. HT29) is measured without and with increasing concentrations of the antibody. The IC₅₀ values of the antibodies according to the invention for the binding of IGF-I and IGF-II to IGF-IR are no more than 2 nM and the ratio of the IC₅₀ values for binding of IGF-I/IGF-II to IGF-IR is about 1:3 to 3:1. IC₅₀ values are measured as average or median values of at least three independent measurements. Single IC₅₀ values may be out of the scope.

The term “inhibiting the binding of IGF-I and IGF-II to IGF-IR” as used herein refers to inhibiting the binding of I¹²⁵-labeled IGF-I or IGF-II to IGF-IR presented on the surface of HT29 (ATCC HTB-38) tumor cells in an in vitro assay. Inhibiting means an IC₅₀ value of 2 nM or lower.

The term “IGF-IR expressing cells” refers to such cells which are overexpressing IGF-I receptor to about at least 20,000 receptors/cell. Such cells are, for example, tumor cell lines such as NCI H322M or HT29, or a cell line (e.g. 3T3 ATCC CRL1658) overexpressing IGF-IR after transfection with an expression vector for IGF-IR. The amount of receptors per cell is measured according to Lammers, R., et al., EMBO J. 8 (1989) 1369-1375.

The term “inhibiting of IGF-IR phosphorylation” refers to a cellular phosphorylation assay using 3T3 cells providing 400,000 to 600,000 molecules IGF-IR per cell, preferably 1.0 to 1.5 Mio, molecules IGF-IR per cell, in a medium containing 0.5% heat inactivated fetal calf serum (FCS) and 10 nM human IGF-1, when compared to such an assay without said antibody. Phosphorylation is detected by Western blotting using an antibody specific for tyrosine-phosphorylated proteins. Heat inactivation of FCS is performed by short term heating to 56° C. for inactivation of the complement system.

The term “inhibiting of PKB phosphorylation” refers to a cellular phosphorylation assay using 3T3 cells providing 400,000 to 600,000 molecules IGF-IR per cell, preferably 1.0 to 1.5 Mio, molecules IGF-IR per cell, in a medium containing 0.5% heat inactivated fetal calf serum (FCS) and 10 nM human IGF-1, when compared to such an assay without said antibody. Phosphorylation is detected by Western blotting using an antibody specific for PKB phosphorylated at serine 473 of PKB (Akt 1, Swiss Prot Acc. No. P31749).

The term “antibody-dependent cellular cytotoxicity (ADCC)” refers to lysis of human tumor target cells by an antibody according to the invention in the presence of effector cells. ADCC is measured preferably by the treatment of a preparation of IGF-IR expressing cells with an antibody according to the invention in the presence of effector cells such as freshly isolated PBMC or purified effector cells from buffy coats, like monocytes or NK cells.

The term “complete inhibition of IGF-I mediated signal transduction” refers to the inhibition of IGF-I-mediated phosphorylation of IGF-IR. For such an assay, IGF-IR expressing cells, preferably H322M cells, are stimulated with IGF-I and treated with an antibody according to the invention (an antibody concentration of 5 nM or higher is useful). Subsequently, an SDS PAGE is performed and phosphorylation of IGF-IR is measured by Western blotting analysis with an antibody specific for phosphorylated tyrosine. Complete inhibition of the signal transduction is found if on the Western blot visibly no band can be detected which refers to phosphorylated IGF-IR.

The antibodies according to the invention show preferably a binding to the same epitope of IGF-IR as antibody 18 or are inhibited in binding to IGF-IR due to steric hindrance of binding by antibody 18. Binding inhibition can be detected by an SPR assay using immobilized antibody 18 and IGF-IR at a concentration of 20-50 nM and the antibody to be detected at a concentration of 100 nM. A signal reduction of 50% or more shows that the antibody competes with antibody 18. Such an assay can be performed in the same manner by using antibody 22 as an immobilized antibody.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The antibodies according to the invention inhibit IGF-IR phosphorylation of tyrosine and preferably also PKB phosphorylation of serine to a similar extent.

The antibodies according to the invention preferably downregulate the IGF-IR protein level in tumor cells and in tumors, e.g. xenograft tumors.

The antibodies according to the invention inhibit preferably the three-dimensional growth of tumor cells in a colony formation assay as well as proliferation of IGF-IR expressing cells (e.g. NIH 3T3 cells).

The antibodies according to the invention preferably do not inhibit binding of insulin to insulin receptor in a binding competition assay on insulin receptor overexpressing 3T3 cells using the antibody in a concentration of 200 nmol/l.

As used herein, the term host cell covers any kind of cellular system which can be engineered to generate the polypeptides and antigen-binding molecules of the present invention. In one embodiment, the host cell is able and engineered to allow the production of an antigen binding molecule with modified glycoforms. The host cells have been further manipulated to express increased levels of one or more polypeptides having GnTIII activity. CHO cells are preferred as host cells.

For the protein expression in the host cell, nucleic acids encoding light and heavy chains or fragments thereof are inserted into expression vectors by standard methods. Expression is performed in such host cells, and the antibody is recovered from the cells (supernatant or cells after lysis).

The general methods for recombinant production of antibodies are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Arzneimittelforschung Drug Res. 48 (1998) 870-880.

The antibodies may be present in whole cells, in a cell lysate, or in a partially purified or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al. (ed.), Current Protocols in Molecular Biology, Greene Publishing and Wiley Interscience, New York (1987). The control sequences that are suitable for prokaryotes, for example, include a promoter, optionally an operator sequence, and a ribosome binding site. Eukaryotic cells are known to utilize promoters, enhancers and polyadenylation signals.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies is readily isolated and sequenced using conventional procedures. The hybridoma cells can serve as a source of such DNA and RNA.

The invention also pertains to immunoconjugates comprising the antibody according to the invention conjugated to a cytotoxic agent such as a chemotherapeutic agent, toxin (e.g., an enzymatically active toxin of bacterial, fungal, plant or animal origin, or fragments thereof), a radioactive isotope (i.e., a radioconjugate) or a prodrug of a cytotoxic agent. Agents useful in the generation of such immunoconjugates have been described above. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes.

Conjugates of the antibody and cytotoxic agent are made using a variety of bifunctional protein coupling agents such as N-succinimidyl-3-(2-pyridyldithiol) propionate (SPDP), iminothiolane (IT), bifunctional derivatives of imidoesters; (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediatnine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta, E. S., et al., Science 238 (1987) 1098-1104). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026.

In another aspect, the present invention provides a composition, e.g. a pharmaceutical composition, containing an antibody of the present invention, formulated together with a pharmaceutically acceptable carrier.

Pharmaceutical compositions of the invention also can be administered in combination therapy, i.e., combined with other agents like chemotherapeutic or c cytotoxic agents or prodrugs. For example, the combination therapy can include a composition of the present invention with at least one anti-tumor agent or other conventional therapy.

A “chemotherapeutic agent” is a chemical compound useful in the treatment of cancer. Examples of chemotherapeutic agents include Adriamycin, Doxorubicin, 5-Fluorouracil, Cytosine arabinoside (“Ara-C”), Cyclophosphamide, Thiotepa, Taxotere (docetaxel), Busulfan, Gemcitabine, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins (see U.S. Pat. No. 4,675,187), Melphalan and other related nitrogen mustards.

The term “cytotoxic agent” as used herein refers to a substance that inhibits or prevents the function of cells and/or causes destruction of cells. The term is intended to include radioactive isotopes, chemotherapeutic agents, and toxins such as enzymatically active toxins of bacterial fungal, plant or animal origin, or fragments thereof.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, D. E., Biochem. Soc. Trans. 14 (1986) 375-382, and Stella, V. J. and Himmelstein, K. J., Prodrugs: A Chemical Approach to Targeted Drug Delivery, In: Directed Drug Delivery, Borchardt, R. T., et al. (ed.), Humana Press, Clifton, N.J. (1985), pp. 247-267. The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam ring prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral or spinal administration (e.g. by injection or infusion).

A “pharmaceutically acceptable salt” refers to a salt that retains the desired biological activity of the antibody and does not impart any undesired toxicological effects (see e.g. Berge, S. M., et al., J. Pharm. Sci. 66 (1977) 1-19). Such salts are included in the invention. Examples of such salts include acid addition salts and base addition salts. Acid addition salts include those derived from nontoxic inorganic acids, such as hydrochloric salts.

A composition of the present invention can be administered by a variety of methods known in the art. As will be appreciated by the skilled artisan, the route and/or mode of administration will vary depending upon the desired results.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Preferred doses are considerably lower than doses of an antibody produced in a nonglycomodified CHO host cell like CHO DG44. The composition must be sterile and fluid to the extent that the composition is deliverable by syringe. In addition to water, the carrier preferably is an isotonic buffered saline solution.

Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

Preferably a partially fucosylated antibody according to the invention is useful for the treatment of NSCLC in combination with Erlotinib (Tarceva®), for the treatment of breast cancer in combination with Herceptin® (Trastuzumab), and pancreatic tumors in combination with gemcitabine (Gemzar®).

The following examples, figures and sequence listing are provided to aid the understanding of the present invention, the true scope of which is set forth in the appended claims. It is understood that modifications can be made in the procedures set forth without departing from the spirit of the invention.

### EXAMPLES

### Plasmids

The expression system comprises the CMV promoter system (EP 0323997) and is described in tables 1 and 2. As antibody an IgG1 antibody against IGF-1R (WO 2005/005635; AK18 or AK22) can be used.

TABLE 1
pETR 2880 (Antibody expression vector)

| Element | Length | Description |
|---|---|---|
| IR18 HC | 1404 | encoding heavy chain of AK18 or AK22 |
| IR18 LC | 714 | encoding light chain of AK18 or AK22 |
| GS | 1122 | encoding glutamine-synthetase |
| SV40E | 343 | Promoter |
| hCMV promoter | 1142 | Promoter |
| Intron | 947 | Intron |

TABLE 2
pETR 2273 (Glycosylation vector)

| Element | Length | Description |
|---|---|---|
| chimeric MPSV | 875 | Promoter |
| promoter (contains | | |
| enhancer of | | |
| hCMV promoter) | | |
| synthetic intron | 1324 | Intron |
| GnTIII | 1644 | encoding N-acetylglucosaminyl- |
| | | transferase III |
| ManII | 3435 | Encoding mannosidase II |
| pac | 600 | encoding puromycin acetyltransferase |
| | | from Streptomyces alboniger |
| polyA | 49 | polyadenylation signal |

Both plasmids were cotransfected in a glutamine prototroph CHO or HEK293 host cell (EP 0 256 055). The cell line was cultivated as fed batch cultivation for up to 14 days in serum free medium to generate antibody batches with different amounts of fucosylation (samples 1-2, CHO). The antibody was isolated from the supernant and purified by chromatographic methods.

WT antibody (AK18 showing fucosylation of 95%) is recombinantly produced in a Chinese hamster ovarian (CHO) cell line, CHO-DG44 (Flintoff, W. F., et al., Somat. Cell Genet. 2 (1976) 245-261; Flintoff, W. F., et al., Mol. Cell. Biol. 2 (1982) 275-285; Urlaub, G., et al., Cell 33 (1983) 405-412; Urlaub, G., et al., Somat. Cell Mol. Genet. 12 (1986) 555-566). CHO-DG44 cells were grown in MEM alpha Minus Medium (Gibco No. 22561), 10% dialysed FCS (Gibco No. 26400-044) and 2 mmol/L L-Glutamine, 100 μM Hypoxanthin, 16 μM Thymidin (HT supplement).

### Example 1

### Determination of the Affinity of Anti-IGF-IR Antibodies to IGF-IR

### Instrument: BIACORE® 3000

### Chip: CM5

Coupling: amine coupling

### Buffer: HBS (HEPES, NaCl), pH 7.4, 25° C.

For affinity measurements anti human FCγ antibodies (from goat) have been coupled to the chip surface for presentation of the glycol engineered antibody against IGF-IR. IGF-IR extracellular domain was added in various concentrations in solution. Association was measured by an IGF-IR-injection of 2 minutes; dissociation was measured by washing the chip surface with buffer for 3 minutes. The affinity data for antibodies 18 and 22 are shown in Table 3.

TABLE 3
Affinity data measured by SPR (BIACORE ® 3000)

| Antibody | ka (1/Ms) | kd (1/s) | KD (M) |
|---|---|---|---|
| No. 1 | 1.98 × 10⁵ | 2.02 × 10⁻⁴ | 1.02 × 10⁻⁹ |
| No. 2 | 1.86 × 10⁵ | 2.68 × 10⁻⁴ | 1.44 × 10⁻⁹ |

### Example 2

### Determination of Antibody Mediated Effector Functions by Anti-IGF-IR HuMAbs

In order to determine the capacity of the generated HuMAb antibodies to elicit immune effector mechanisms antibody-dependent cell cytotoxicity (ADCC) studies were performed. To study the effects of the antibodies in ADCC, H322M, DU145 or other suitable IGF-IR expressing cells (1×10⁶ cells/ml) were labeled with 1 μl per ml BATDA solution (Perkin Elmer) for 25 minutes at 37° C. in a cell invubator. Afterwards, cells were washed four times with 10 ml of RPMI-FM/PenStrep and spun down for 10 minutes at 200×g. Before the last centrifugation step, cell numbers were determined and cells diluted to 1×10⁵ cells/ml in RPMI-FM/PenStrep medium from the pellet afterwards. The cells were plated 5,000 per well in a round bottom plate, in a volume of 50 μl. HuMAb antibodies were added at a final concentration ranging from 25-0.1 μg/ml in a volume of 50 μl cell culture medium to 50 μl cell suspension. Subsequently, 50 μl of effector cells, freshly isolated PBMC were added at an E:T ratio of 25:1. The plates were centrifuged for 1 minutes at 200×g, followed by an incubation step of 2 hours at 37° C. After incubation the cells were spun down for 10 minutes at 200×g and 20 μl of supernatant was harvested and transferred to an Optiplate 96-F plate. 200 μl of Europium solution (Perkin Elmer, at room temperature) were added and plates were incubated for 15 minutes on a shaker table. Fluorescence is quantified in a time-resolved fluorometer (Victor 3, Perkin Elmer) using the Eu-TDA protocol from Perkin Elmer.

The magnitude of cell lysis by ADCC is expressed as % of the maximum release of TDA fluorescence enhancer from the target cells lysed by detergent corrected for spontaneous release of TDA from the respective target cells. Results are shown in table 4:

TABLE 4

| Sample | Specific tumor cell lysis |
|---|---|
| No. 1 | 66% |
| No. 2 | 84% |

### Example 3

### Analysis of Glycostructure of Antibody

For determination of the relative ratios of fucose- and non-fucose (a-fucose) containing oligosaccharide structures, released glycans of purified antibody material were analyzed by MALDI-Tof-mass spectrometry. For this, the antibody sample (about 50 μg) was incubated over night at 37° C. with 5 mU N-Glycosidase F (Prozyme# GKE-5010B) in 0.1M sodium phosphate buffer, pH 6.0, in order to release the oligosaccharide from the protein backbone. Subsequently, the glycan structures released were isolated and desalted using NuTip-Carbon pipet tips (obtained from Glygen: NuTip1-10 μl, Cat.Nr#NT1CAR). As a first step, the NuTip-Carbon pipet tips were prepared for binding of the oligosaccharides by washing them with 3 μL 1M NaOH followed by 20 μL pure water (e.g. HPLC-gradient grade from Baker, # 4218), 3 μL 30% v/v acetic acid and again 20 μl pure water. For this, the respective solutions were loaded onto the top of the chromatography material in the NuTip-Carbon pipet tip and pressed through it. Afterwards, the glycan structures corresponding to 10 μg antibody were bound to the material in the NuTip-Carbon pipet tips by pulling up and down the N-Glycosidase F digest described above four to five times. The glycans bound to the material in the NuTip-Carbon pipet tip were washed with 20 μL pure water in the way as described above and were eluted stepwise with 0.5 μL 10% and 2.0 μL 20% acetonitrile, respectively. For this step, the elution solutions were filled in a 0.5 mL reaction vials and were pulled up and down four to five times each. For the analysis by MALDI-T of mass spectrometry, both eluates were combined. For this measurement, 0.4 μL of the combined eluates were mixed on the MALDI target with 1.6 μL SDHB matrix solution (2.5-Dihydroxybenzoic acid/2-Hydroxy-5-Methoxybenzoic acid [Bruker Daltonics #209813] dissolved in 20% ethanol/5 mM NaCl at 5 mg/ml) and analysed with a suitably tuned Bruker Ultraflex TOF/TOF instrument. Routinely, 50-300 shots were recorded and summed up to a single experiment. The spectra obtained were evaluated by the flex analysis software (Bruker Daltonics) and masses were determined for the each of the peaks detected. Subsequently, the peaks were assigned to fucose or a-fucose (non-fucose) containing glyco structures by comparing the masses calculated and the masses theoretically expected for the respective structures (e.g. complex, hybrid and oligo- or high-mannose, respectively, with and without fucose).

For determination of the ratio of hybrid structures, the antibody sample was digested with N-Glycosidase F and Endo-Glycosidase H concomitantly. N-glycosidase F releases all N-linked glycan structures (complex, hybrid and oligo- and high mannose structures) from the protein backbone and the Endo-Glycosidase H cleaves all the hybrid type glycans additionally between the two GlcNAc-residue at the reducing end of the glycan. This digest was subsequently treated and analysed by MALDI-T of mass spectrometry in the same way as described above for the N-Glycosidase F digested sample. By comparing the pattern from the N-Glycosidase F digest and the combined N-glycosidase F/Endo H digest, the degree of reduction of the signals of a specific glyco structure is used to estimate the relative content of hybrid structures.

The relative amount of each glyco structure was calculated from the ratio of the peak height of an individual glycol structure and the sum of the peak heights of all glyco structures detected. The relative amount of afucose is the percentage of fucose-lacking structures related to all glyco structures identified in the N-Glycosidase F treated sample (e.g. complex, hybrid and oligo- and high-mannose structures, resp.), see table 5.

TABLE 5

| Sample | A-fucosylation (in %) |
|---|---|
| No. 1 | 64% |
| No. 2 | 61% |
| No. 3 (HEK293) | 79% |

### Example 4

### Determination of the Affinity of Anti-IGF-IR Antibodies to FcgR III (CD16a)

His-CD16a was amine-coupled to the surface of a CM5-chip (˜660 RU).

### Instrument: Biacore3000

Running and dilution buffer: HBS-P
Measurement: 1 data point/second at 25° C.; 5 min injection of antibody at 100 nM; 5 min dissociation at a flow rate of 50 μl/min; samples pre-cooled at 12° C.; regeneration of the surface with 7.5 mM NaOH/1 M NaCl for 1 min. Results: RU for wt: 11; RU for 45% fucosylation: 65; RU for 60% fucosylation: 72

### Example 5

### Toxicity Study

A two week toxicity study in Cynomolgus monkeys was performed. The partially fucosylated antibody was administered at days 1, 4, 7, 11 in a dose of 10 mg/kg/day. In comparison to wt antibody no drug related changes were observed in general condition and behavior, body weight, food consumption, hematology, ECG, clinical chemistry (excludingALAT), macroscopy, or organ weights.

1. An antibody that binds to IGF-IR that comprises a human heavy chain constant domains, wherein the heavy chain constant domains are glycosylated with a sugar chain at Asn297, wherein said sugar chain comprises between 20% and 50% fucose.

2. The antibody according to claim 1, wherein said sugar chain comprises between 20% and 40% fucose.

3. The antibody according to claim 1 wherein if the sugar chain comprises N-glycolylneuraminic acid, the amount of N-glycolylneuraminic acid is 1% or less of the sugar chain and wherein if the sugar chain comprises N-terminal alpha-1,3-galactose, the amount of N-terminal alpha-1,3-galactose is 1% or less of the sugar chain.

4. The antibody according to claim 3, wherein the amount of NGNA is 0.5% or less.

5. The antibody according to claim 3, wherein the amount of N-terminal alpha 1,3 galactose is 0.5% or less.

6. The antibody according to claim 1, wherein the antibody is a chimeric, humanized or human antibody.

7. The antibody according to claim 1, wherein the antibody shows one or more properties selected from the group consisting of:
a) shows a ratio of IC₅₀ values of inhibition of the binding of IGF-I to IGF-IR to the inhibition of binding of IGF-II to IGF-IR of 1:3 to 3:1;
b) inhibits for at least 80%, preferably at least 90%, at a concentration of 5 nM IGF-IR phosphorylation in a cellular phosphorylation assay using HT29 cells in a medium containing 0.5% heat inactivated fetal calf serum (FCS) and 10 nM human IGF-1, when compared to such an assay without said antibody; and
c) shows no IGF-IR stimulating activity (no signaling, no IGF-1 mimetic activity) measured as PKB phosphorylation at a concentration of 10 μM in a cellular phosphorylation assay using 3T3 cells providing 400,000 to 600,000 molecules IGF-IR per cell in a medium containing 0.5% heat inactivated fetal calf serum (FCS) when compared to such an assay without said antibody.

8. The antibody according to claim 3, wherein said antibody has an affinity for IGFI-IR of about 10⁻¹³ to 10⁻⁹ M (K_{D}).

9. The antibody according to claim 1, wherein said antibody comprises:
a) an antibody heavy chain comprising as CDRs CDR1 (aa 31-35), CDR2 (aa 50-66) and CDR3 (aa 99-107) of SEQ ID NO:1 or 3; and
b) an antibody light chain comprising as CDRs CDR1 (aa 24-34), CDR2 (aa 50-56) and CDR3 (aa 89-98) of SEQ ID NO:2 or 4.

10. A pharmaceutical composition comprising an antibody according to claim 1 and a pharmaceutically acceptable carrier or excipient.

11. Method for the treatment of a patient in need of an antitumor therapy, comprising administering to the patient an effective amount of an antibody according to claim 1.

12. The method according to claim 11, wherein the antibody is administered in combination with a cytotoxic agent, a prodrug thereof or cytotoxic radiotherapy.

13. A CHO cell that recombinantly expresses the GnTIII protein and an antibody according to claim 1.

14. An antibody according the claim 1, wherein said antibody has a high binding affinity for the FcγRIII.

15. A method according to claim 11, wherein said patient in need of antitumor therapy is suffering from a member selected from the group consisting of breast cancer, pancreatic cancer and non-small cell lung cancer.
